(19) United States
(12) Patent Application Publication
(10) Pub. No.: US 2022/0215960 A1
(43) Pub. Date: Jul. 7, 2022

(54) AUXILIARY METHOD FOR DIAGNOSIS OF LOWER URINARY TRACT SYMPTOMS

(71) Applicant: SAMSUNG LIFE PUBLIC WELFARE FOUNDATION, Seoul (KR)

(72) Inventors: Kyu Sung LEE, Seoul (KR); Deok Hyun HAN, Seoul (KR); Baek Hwan CHO, Seoul (KR); Kyeong Won CHO, Seoul (KR)

(21) Appl. No.: 17/603,818

(22) PCT Filed: Feb. 12, 2020
(86) PCT No.: PCT/KR2020/001982

§ 371 Date: Nov. 18, 2021

(30) Foreign Application Priority Data
Apr. 17, 2019 (KR) 10-2019-0044896

(51) Int. Cl. G16H 50/20

(52) U.S. Cl. CPC G16H 50/20

(57) ABSTRACT

A method for supporting diagnosis of LUTS may include: receiving urinary system data of an examinee; deriving prediction result data for the examinee by using an LUTS prediction model for the received urination system data; and providing the derived prediction result data to a user terminal.

### FIELD OF THE INVENTION

The present disclosure relates to a method for supporting diagnosis of LUTS (Lower Urinary Tract Symptom), and more particularly, to a method for supporting diagnosis of LUTS, which can derive prediction result data for an examinee by using an LUTS prediction model for urinary system data of the examinee, and provide the derived prediction result data to a user (e.g. doctor), thereby supporting diagnosis.

### BACKGROUND OF THE INVENTION

LUTS collectively represents various symptoms which appear during urine storage and voiding phases, for example, difficulty in starting stream, residual urine, frequent urination, weak stream, straining during urination, nocturnal enuresis, urinary urgency, intermittent urination and the like. Recently, as the social conditions gradually become complicated while the consumption of animal fat increases, more and more people suffer from the LUTS, due to various reasons such as mental stress, smoking, drinking, weight gain, lack of rest and lack of exercise. When the LUTS of a patient develops, the patient's activity is limited, and the patient is always exposed to an anxiety state and tension state, which causes mental stress. When a patient suffers from a lack of sleep because the patient frequently goes to the bathroom in the nighttime, physical fatigue is aggravated to cause various physical problems.

However, it is difficult to not only accurately diagnose the LUTS only with symptoms, but also efficiently treat the LUTS only with drug and surgery. The LUTS is diagnosed through UDS (Urodynamic Study) for evaluating the function of the bladder. In particular, the UDS has been mainly carried out to decide whether to perform prostatic surgery, or carried out to distinguish between a patient having only DUA (Detrusor Under-Activity) which is not effectively treated through surgery and a patient having BOO (Bladder Outlet Obstruction) which is known to be effectively treated through surgery.

The UDS includes a process of inserting a pressure measurement pipe into the bladder and anus of a patient, measuring pressure while slowly filling the bladder with saline solution, and then measuring the pressure of the bladder while the patient urinates. That is, since the UDS for diagnosing the LUTS may make the patient uncomfortable and embarrassed and is performed with a catheter inserted into the bladder and anus for a long time, the patient may be exposed to the risk of infection, and feel pain and shame.

Therefore, there is a continuous demand for the development of a method for supporting diagnosis of LUTS, which can relieve the pain and shame of an examinee, and accurately determine the state of the urinary system of the examinee.

### RELATED ART DOCUMENT

[Patent Document]
Korean Patent Application Publication No. 10-2007-0074288 (published on Jul. 12, 2007).

### SUMMARY OF THE INVENTION

### Technical Problem

Various embodiments are directed to a method for supporting diagnosis of LUTS (Lower Urinary Tract Symptom), which can minimize physical/mental pain of an examinee by using only noninvasive test result data, and automatically predict the type of LUTS.

Also, various embodiments are directed to a method for supporting diagnosis of LUTS, which provides information on whether USD is needed, such that the USD is not performed for an examinee who is unlikely to have BOO, thereby reducing the time and cost.

### Technical Solution

In an embodiment, a method for supporting diagnosis of LUTS (lower urinary tract symptom) may be provided.

The method for supporting diagnosis of LUTS may include: receiving urinary system data of an examinee; deriving prediction result data for the examinee by using an LUTS prediction model for the received urination system data; and providing the derived prediction result data to a user terminal.

The method may further include: learning the correlation between diagnosis result data and urinary system data acquired in advance and stored in a database, through a machine learning algorithm; and generating an LUTS prediction model on the basis of the learning result.

The urination system data may include one or more of the age, number of urinations, residual urine volume, uroflowmetry index, prostate symptom score, past medical history and voiding efficacy of the examinee, and the prediction result data may include one or more of predicted diagnosis, BOO (Bladder Outlet Obstruction) probability, DUA (Detrusor Under-Activity) probability, and information on whether UDS (Urodynamic Study) is needed.

The LUTS prediction model may further include a first neural network for predicting the degree of BOO and a second neural network for predicting the degree of DUA.

The LUTS prediction model may be formed so that the second neural network has an output of the first neural network as an input value thereof, or the first neural network has an output of the second neural network as an input value thereof.

The machine learning algorithm may include any one of ANN (Artificial Neural Network), stacked auto-encoder, DNN (Deep Neural Network) and LSTM (Long Short Term Memory).

In another embodiment, an LUTS diagnosis supporting system may be provided.

The LUTS diagnosis supporting system may include: a data input unit configured to receive urinary system data of an examinee; a diagnosis prediction unit configured to derive prediction result data for the examinee by using an LUTS prediction model for the received urinary system data; and a result providing unit configured to provide the derived prediction result data to a user terminal.

The LUTS diagnosis supporting system may further include a prediction model generation unit configured to learn the correlation between diagnosis result data and urinary system data acquired in advance and stored in a database, through a machine learning algorithm, and generate an LUTS prediction model on the basis of the learning result.

The urination system data may include one or more of the age, number of urinations, residual urine volume, uroflowmetry index, prostate symptom score, past medical history and voiding efficacy of the examinee, and the prediction result data may include one or more of predicted diagnosis, BOO (Bladder Outlet Obstruction) probability, DUA (Detrusor Under-Activity) probability, and information on whether UDS (Urodynamic Study) is needed.

The LUTS prediction model may further include a first neural network for predicting the degree of BOO and a second neural network for predicting the degree of DUA.

The LUTS prediction model may be formed so that the second neural network has an output of the first neural network as an input value thereof, or the first neural network has an output of the second neural network as an input value thereof.

The machine learning algorithm may include any one of ANN (Artificial Neural Network), stacked auto-encoder, DNN (Deep Neural Network) and LSTM (Long Short Term Memory).

In another embodiment, a computer readable recording medium in which a program for implementing the above-described is recorded may be provided.

### Advantageous Effects

In accordance with the embodiments of the present disclosure, it is possible to minimize physical/mental pain of an examinee by using only noninvasive test result data, and automatically predict the type of LUTS.

Furthermore, it is possible to provide information on whether USD is needed, such that the USD is not performed for an examinee who is unlikely to have BOO, thereby reducing the time and cost.

### BRIEF DESCRIPTION OF DRAWINGS

FIGS. 1 and 2 are flowchart illustrating a method for supporting diagnosis of LUTS (Lower Urinary Tract Symptom) in accordance with an embodiment of the present disclosure.

FIG. 3 is a block diagram illustrating an LUTS diagnosis supporting system in accordance with an embodiment of the present disclosure.

FIG. 4 is a diagram illustrating an example of the LUTS diagnosis supporting system in accordance with the embodiment of the present disclosure.

FIG. 5 is a block diagram illustrating an LUTS prediction model in the LUTS diagnosis supporting system in accordance with the embodiment of the present disclosure.

FIGS. 6A and 6B are each a block diagram illustrating an LUTS prediction model which is formed when an output of a first neural network (or second neural network) is applied as an input of the second neural network (or first neural network), in the LUTS diagnosis supporting system in accordance with the embodiment of the present disclosure.

FIGS. 7A and 7B are each a diagram illustrating an example of the LUTS prediction model which is formed when an output of the first neural network (or second neural network) is applied as an input of the second neural network (or first neural network), in the LUTS diagnosis supporting system in accordance with the embodiment of the present disclosure.

FIGS. 8 and 9 are diagrams illustrating an example of a user terminal which displays information provided through a result providing unit in the LUTS diagnosis supporting system in accordance with the embodiment of the present disclosure.

### DETAILED DESCRIPTION OF THE INVENTION

Hereafter, exemplary embodiments of the present disclosure will be described in detail with reference to the accompanying drawings, such that the present disclosure can be easily carried out by those skilled in the art to which the present disclosure pertains. However, the present disclosure can be embodied in various forms, and are not limited to the embodiments described herein. In the drawings, components which have nothing to do with the description will be omitted in order to clearly describe the present disclosure. Throughout the specification, like components will be represented by like reference numerals.

The terms used in this specification will be briefly described, and then the present disclosure will be described in detail.

General terms which are used as widely as possible at the moment are selected as the terms used herein in consideration of functions in the present disclosure. However, the terms may be changed depending on a technician's intention, a precedent or an appearance of new technique. In a specific case, a term randomly selected by the applicant may be used. In this case, the meaning of the term will be described in detail in the corresponding part of this specification. Therefore, the terms used herein should not be defined by the names of the terms, but defined by the meanings of the terms based on the overall disclosures set forth herein.

Throughout the specification, when an element “includes” a component, it indicates that the element does not exclude another component unless specifically described to the contrary, but can further include another component. The terms such as “ . . . unit” and “module” in this specification may indicate a unit for processing one or more functions or operations, and the unit may be embodied in hardware, software or a combination of hardware and software. Throughout the specification, when one element is referred to as being ‘connected to’ or ‘coupled to’ another element, it not only indicates that the former element is directly connected or coupled to the latter element, but also indicates that the former element is connected or coupled to the latter element with another element interposed therebetween.

Hereafter, the present disclosure will be described in detail with reference to the drawings.

As an embodiment of the present disclosure, a method for supporting diagnosis of LUTS (Lower Urinary Tract Symptom) may be provided. Furthermore, the method for supporting diagnosis of LUTS may be performed by an LUTS diagnosis supporting system 10 which will be described below. Hereafter, the method for supporting diagnosis of LUTS, which is performed by the LUTS diagnosis supporting system 10, will be described.

In this specification, the LUTS collectively represents various symptoms which are likely to occur in the urinary system. Examples of the LUTS may include various symptoms such as difficulty in starting stream, residual urine, frequent urination, weak stream, straining during urination, nocturnal enuresis, urinary urgency, intermittent urination and the like, which occur during urine storage and voiding phases. The urinary system may include organisms such as the kidneys, ureters, bladder and urethra.

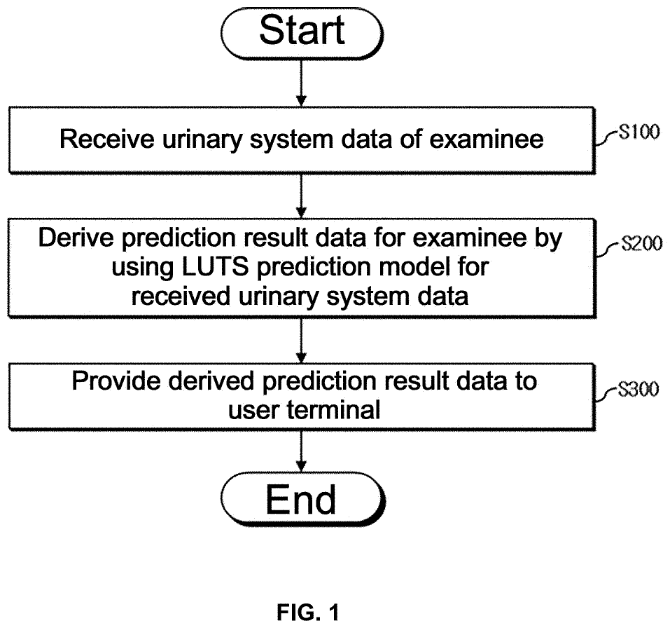

FIG. 1 is a flowchart illustrating a method for supporting diagnosis of LUTS in accordance with an embodiment of the present disclosure.

Referring to FIG. 1, the method for supporting diagnosis of LUTS in accordance with the embodiment of the present disclosure may include step S100 of receiving urinary system data 20 of an examinee, step S200 of deriving prediction result data 30 for the examinee by using an LUTS prediction model 410 for the received urinary system data 20, and step S300 of providing the derived prediction result data 30 to a user terminal 80.

Furthermore, in the method for supporting diagnosis of LUTS in accordance with the embodiment of the present disclosure, the urinary system data 20 of an examinee may indicate data acquired from the examinee in order to diagnose the LUTS, and include one or more of the age, urination pattern (e.g. number of urinations, residual urine volume, number of urinary urgencies, and number of night urinations), uroflowmetry index, prostate symptom score, past medical history and voiding efficacy of the examinee. The urinary system data 20 may further include a survey result for LUTS diagnosis.

The voiding efficacy may be derived through Equation 1 below.

$$v e = \frac{v v}{v v + r u v} \qquad [ Equation 1 ]$$

where ve represents voiding efficacy, vv represents voided volume, and ruv represents residual urine volume.

That is, the voiding efficacy in this specification indicates a value obtained by dividing the voided volume by the sum of the voided volume and the residual urine volume as in Equation 1. The voiding efficacy may also be included in the urinary system data 20 of the examinee as described above, and utilized for training an LUTS prediction model 410 as will be described below.

Furthermore, the urinary system data 20 may further include image feature data. The image feature data may be acquired through a third neural network trained for urinary system image data. The urinary system image data may include a transrectal ultrasonography image and a multi-parametric MRI image. The third neural network may correspond to CNN (Convolutional Neural Network), DNN (Deep Neural Network) or the like. That is, as described above, the image feature data extracted/acquired from the urinary system image data through the third neural network such as the CNN may be included in the urinary system data 20, and utilized for training the LUTS prediction model 410 as will be described below.

The prediction result data 30 may indicate a result obtained by performing prediction diagnosis through the LUTS prediction model 410, and include one or more of predicted diagnosis, BOO (Bladder Outlet Obstruction) probability, DUA (Detrusor Under-Activity) probability, and information on whether UDS (Urodynamic Study) is needed. That is, the prediction result data 30 may include not only data for sorting LUTS classes (e.g. BOO, DUA and normal) which are determined from the urinary system data 20 of the examinee, but also data on predicted diagnosis results such as the respective probabilities of the sorted classes and information on whether the UDS is needed.

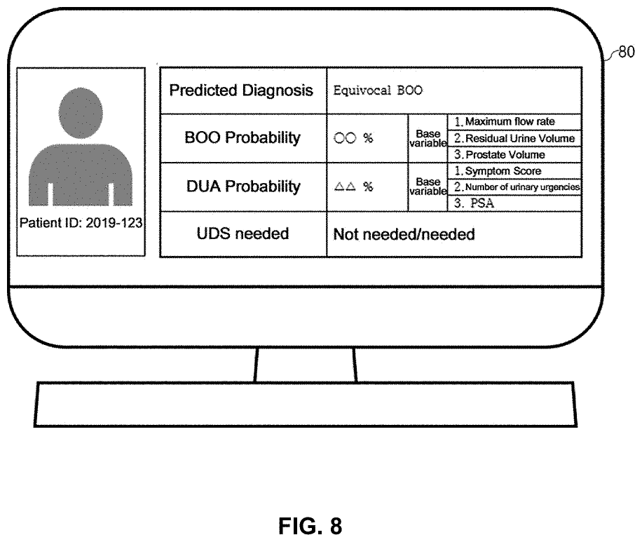

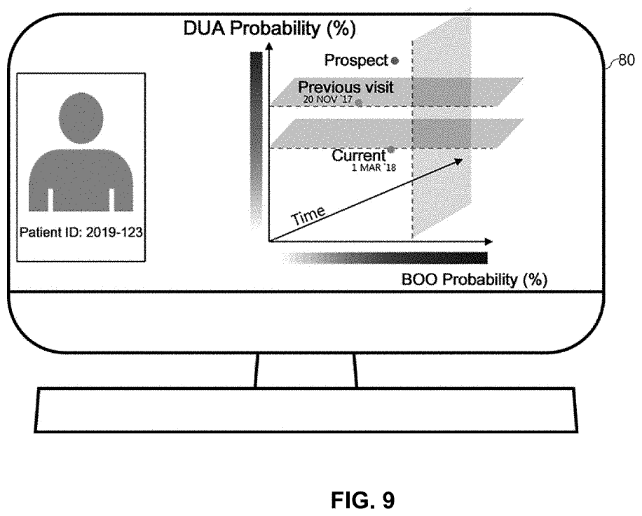

FIGS. 8 and 9 are diagrams illustrating an example of a user terminal 80 which displays information provided through a result providing unit in the LUTS diagnosis supporting system 10 in accordance with the embodiment of the present disclosure.

As illustrated in FIGS. 8 and 9, the above-described prediction result data 30 may be provided and displayed on the user terminal 80. That is, the user terminal 80 indicates a device which can receive the prediction result data 30 and display the prediction result data 30 to a user. The user terminal 80 may be subsidiarily utilized when the user diagnoses the LUTS. Examples of the user terminal 80 may include various types of devices such as a personal PC, smart phone and tablet PC.

In this specification, the user may indicate a medical examiner or medical personnel such as a doctor, who diagnoses the LUTS for an examinee, and the examinee may include not only a patent who suffers from the LUTS, but also an ordinary person who suspects the LUTS and thus wants to check his/her urinary system.

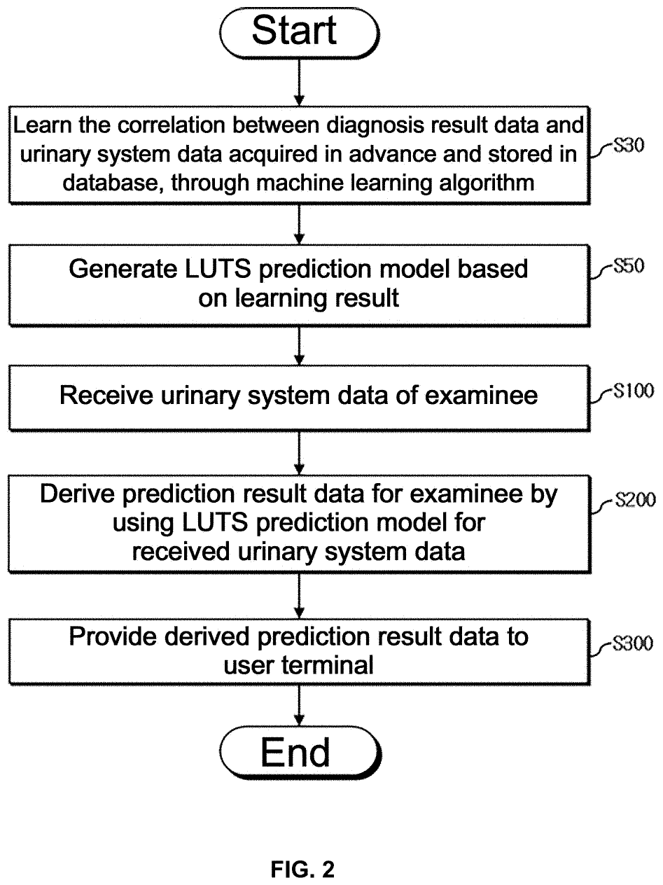

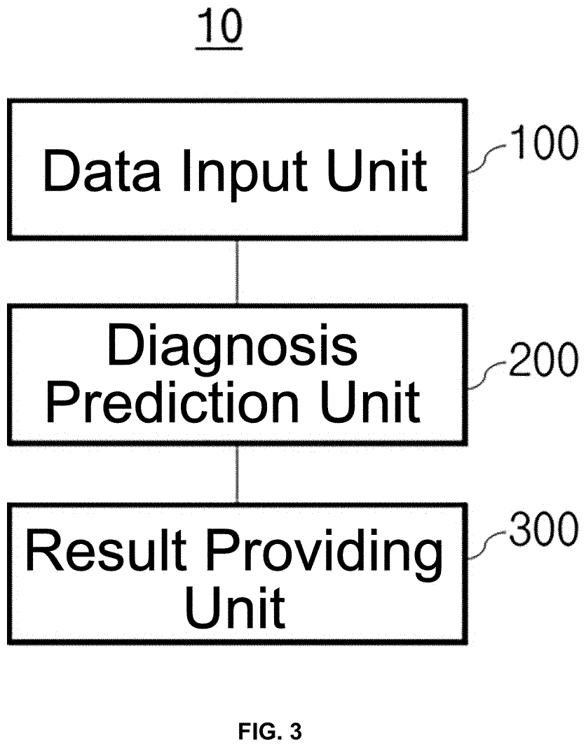

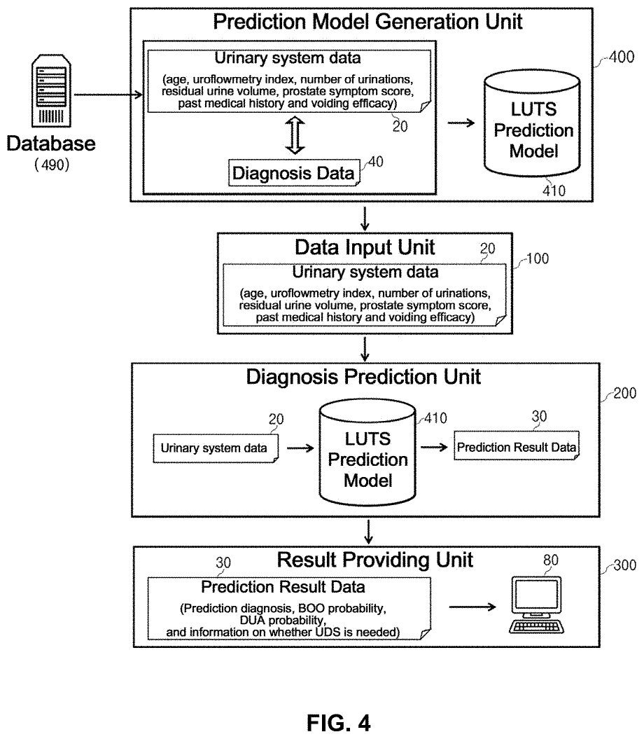

FIG. 2 is a flowchart illustrating the method for supporting diagnosis of LUTS in accordance with the embodiment of the present disclosure, FIG. 3 is a block diagram illustrating the LUTS diagnosis supporting system in accordance with the embodiment of the present disclosure, and FIG. 4 is a diagram illustrating an example of the LUTS diagnosis supporting system 10 in accordance with the embodiment of the present disclosure.

Referring to FIGS. 2 to 4, the method for supporting diagnosis of LUTS in accordance with the embodiment of the present disclosure further include step S30 of learning the correlation between the diagnosis result data 40 and the urinary system data 20 acquired in advance and stored in a database 490, through a machine learning algorithm, and step S50 of generating the LUTS prediction model 410 on the basis of the learning result, before the above-described steps S100, S200 and S300.

That is, in the method for supporting diagnosis of LUTS in accordance with the embodiment of the present disclosure, the LUTS prediction model 410 may be generated through the process of steps S30 and S50.

First, the correlation between the diagnosis result data 40 and the urinary system data 20 acquired in advance and stored in the database 490 may be learned through the machine learning algorithm in step S30.

The database 490 may indicate a data storage place for storing medical data of a patient or examinee as well as the urinary system data 20, and include medical data search systems such as CDW (Clinical Data Warehouse) and PACS (Picture Archiving and Communication System). That is, in the present specification, the database 490 may correspond to a server for storing not only medical records of a doctor for examinees, but also overall data related to examinees, such as various test results.

That is, the correlation between the diagnosis result data 40 and the urinary system data 20 stored in advance in the database 490 may be learned, the urinary system data 20 including the age, urination pattern (e.g. number of urinations, residual urine volume, number of urinary urgencies, and number of night urinations), uroflowmetry index, prostate symptom score, past medical history and voiding efficacy of the examinee. The diagnosis result data 40 may include BOOI (BOO Index), BCI (Bladder Contractility Index) and the like, which are acquired through a UDS for the examinee having the corresponding urinary system data 20 or a diagnosis result of a medical expert for the examinee. The diagnosis result data 40 may additionally include uroflowmetry data, bladder ultrasound image data, penile cuff test data and the like.

In the method for supporting diagnosis of LUTS in accordance with the embodiment of the present disclosure, the machine learning algorithm may include any one of ANN (Artificial Neural Network), stacked auto-encoder, DNN (Deep Neural Network) and LSTM (Long Short Term Memory). However, this is only an example, and various machine learning algorithms or deep learning algorithms except the above-described algorithm may be used. Furthermore, the machine learning/deep learning algorithms may be coupled or connected to form a new prediction model.

In order to overcome the imbalance between a positive sample and a negative sample of the urinary system data 20 in the learning process of step S30, a data mining process may be additionally performed. The data mining process may include a bootstrap aggregating process. That is, through the bootstrap aggregating process, a plurality of bootstrap data may be generated for the urinary system data 20, and modeled and coupled to calculate a model. The bootstrap data may indicate a plurality of sample data having the same size, which are extracted from raw data through random sampling.

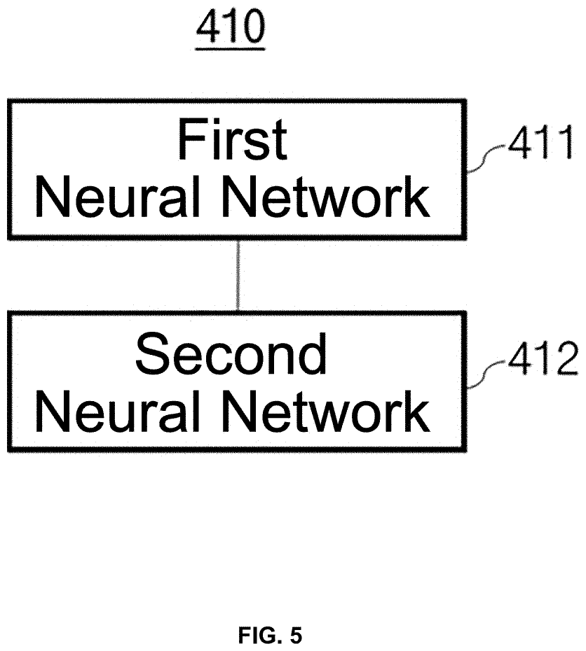

FIG. 5 is a block diagram illustrating the LUTS prediction model 410 in the LUTS diagnosis supporting system 10 in accordance with the embodiment of the present disclosure.

Referring to FIG. 5, the LUTS prediction model 410 in the LUTS diagnosis supporting system in accordance with the embodiment of the present disclosure may further include a first neural network 411 for predicting the degree of BOO and a second neural network 412 for predicting the degree of DUA. However, the LUTS prediction model 410 is not limited to the first and second neural networks 411 and 412, but may be configured as a single neural network for classifying the LUTS into BOO, DUA, combination of BOO and DUA and normal.

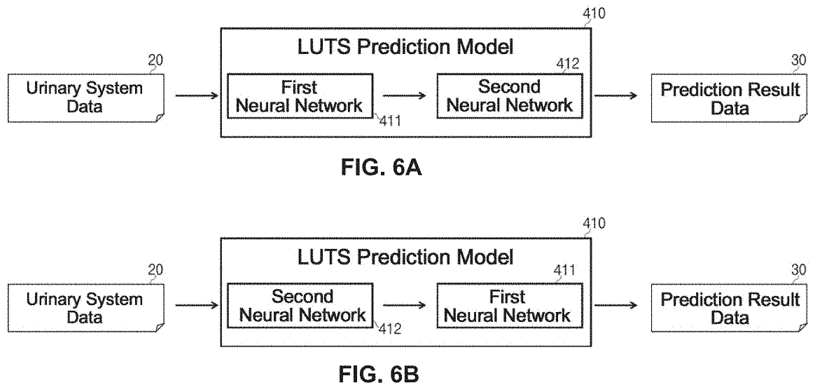

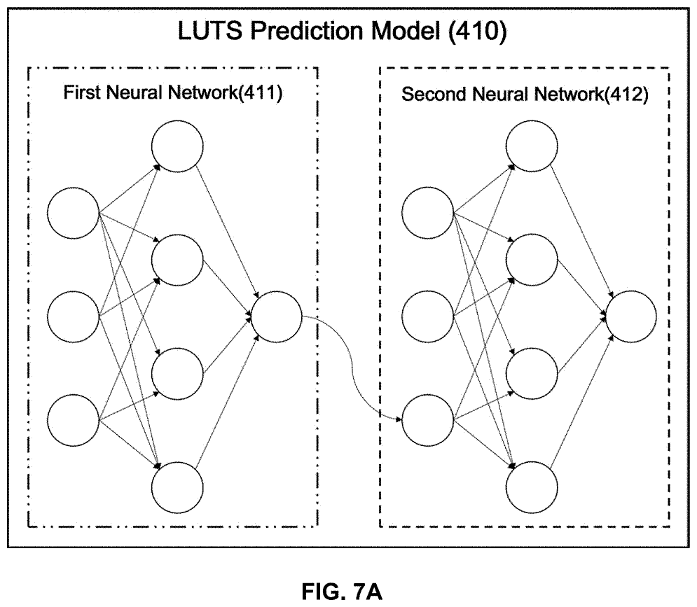

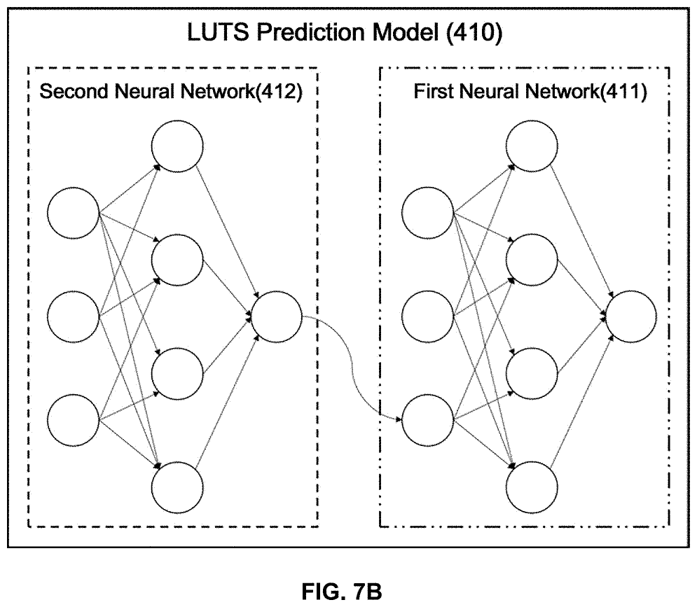

FIGS. 6A and 6B are each a block diagram illustrating the LUTS prediction model 410 which is formed when an output of the first neural network (or second neural network) is applied as an input of the second neural network (or first neural network), in the LUTS diagnosis supporting system 10 in accordance with the embodiment of the present disclosure, and FIGS. 7A and 7B are each a diagram illustrating an example of the LUTS prediction model 410 which is formed when an output of the first neural network (or second neural network) is applied as an input of the second neural network (or first neural network), in the LUTS diagnosis supporting system 10 in accordance with the embodiment of the present disclosure.

FIGS. 6A, 6B, 7A and 7B, the LUTS prediction model 410 in the LUTS diagnosis supporting method in accordance with the embodiment of the present disclosure may be formed so that the second neural network 412 has an output of the first neural network 411 as an input value thereof, or the first neural network 411 has an output of the second neural network 412 as an input value thereof.

That is, in order to improve the prediction possibility or accuracy of the LUTS prediction model 410, the LUTS prediction model 410 may be generated to have the structures described with reference to the above contents and FIGS. 6A, 6B, 7A and 7B.

The LUTS prediction model 410 of FIG. 6A may be formed as an output of the first neural network 411 is applied as an input of the second neural network 412, and have the structure illustrated in FIG. 7A. Similarly, the LUTS prediction model 410 of FIG. 6B may be formed as an output of the second neural network 412 is applied as an input of the first neural network 411, and have the structure illustrated in FIG. 7B.

As an embodiment of the present disclosure, the LUTS diagnosis supporting system 10 may be provided. Hereafter, the contents for the above-described method may be applied to the LUTS diagnosis supporting system 10 which will be described below. Therefore, the descriptions of the same contents as the above-described contents in relation to the system will be omitted herein.

The LUTS diagnosis supporting system 10 in accordance with an embodiment of the present disclosure may include a data input unit 100 configured to receive urinary system data 20 of an examinee, a diagnosis prediction unit 200 configured to derive prediction result data 30 for the examinee by using an LUTS prediction model 410 for the received urinary system data 20, and a result providing unit 300 configured to provide the derived prediction result data 30 to a user terminal 80.

The LUTS diagnosis supporting system 10 in accordance with an embodiment of the present disclosure may further include a prediction model generation unit 400 configured to learn the correlation between diagnosis result data 40 and the urinary system data 20 acquired in advance and stored in a database 490, through a machine learning algorithm, and generate the LUTS prediction model 410 on the basis of the learning result.

In the LUTS diagnosis supporting system 10 in accordance with an embodiment of the present disclosure, the urinary system data 20 may include one or more of the age, the number of urinations, residual urine volume, uroflowmetry index, prostate symptom score, past medical history and voiding efficacy of the examinee, and the prediction result data 30 may include one or more of predicted diagnosis, BOO probability, DUA probability and information on whether UDS is needed.

In the LUTS diagnosis supporting system 10 in accordance with the embodiment of the present disclosure, the LUTS prediction model 410 may further include a first neural network for predicting the degree of BOO and a second neural network for predicting the degree of DUA.

In the LUTS diagnosis supporting system 10 in accordance with the embodiment of the present disclosure, the LUTS prediction model 410 may be formed so that the second neural network has an output of the first neural network as an input value thereof, or the first neural network has an output of the second neural network as an input value thereof.

In the LUTS diagnosis supporting system 10 in accordance with the embodiment of the present disclosure, the machine learning algorithm may include any one of ANN (Artificial Neural Network), stacked auto-encoder, DNN (Deep Neural Network) and LSTM (Long Short Term Memory).

As an embodiment of the present disclosure, a readable recording medium may be provided to a computer in which a program for implementing the above-described method is recorded.

The above-described method may be created as a program which can be executed in a computer, and implemented in a general-purpose digital computer that operates the program using a computer-readable medium. The structure of data used in the above-described method may be recorded into a computer readable medium through various units. However, it should not be understood that the recording medium for recording an executable computer program or code for performing various methods of the present disclosure includes temporary targets such as carrier waves or signals. The computer-readable medium may include storage media such as magnetic storage media (ex. ROM, floppy disk, hard disk and the like) and optical readable media (ex. CD ROM, DVD and the like).

The aforementioned descriptions of the present disclosure are only examples, and those skilled in the art to which the present disclosure pertains can understand that the present disclosure can be easily modified into other specific forms without changing the technical spirit or necessary features of the present disclosure. Therefore, it should be understood that the above-described embodiments are only examples in all aspects and are not limitative. For example, components described in a singular form may be distributed and embodied. Similarly, distributed components may be embodied in a combined form.

The scope of the present disclosure is defined by the following claims rather than the detailed descriptions, and it should be construed that the meaning and scope of the claims and all changes or modifications derived from the equivalents thereof are included in the scope of the present disclosure.

Accordingly, the embodiments disclosed in the present disclosure are intended not to limit but to describe the technical spirit of the present disclosure, and the scope of the technical spirit of the present disclosure is not limited by the embodiments. The scope of the present disclosure shall be interpreted on the basis of the following claims, and it shall be interpreted that all the technical spirit within the scope equivalent thereto falls within the scope of the present disclosure.

1. A method for supporting diagnosis of LUTS (lower urinary tract symptom), comprising:
receiving urinary system data of an examinee;
deriving prediction result data for the examinee by using an LUTS prediction model for the received urination system data; and
providing the derived prediction result data to a user terminal.

2. The method of claim 1, further comprising:
learning a correlation between diagnosis result data and urinary system data acquired in advance and stored in a database, through a machine learning algorithm; and
generating an LUTS prediction model based on a result of the learning.

3. The method of claim 1, wherein the urination system data comprises one or more of an age, number of urinations, residual urine volume, uroflowmetry index, prostate symptom score, past medical history and voiding efficacy of the examinee,
wherein the prediction result data comprises one or more of predicted diagnosis, BOO (Bladder Outlet Obstruction) probability, DUA (Detrusor Under-Activity) probability, and information on whether UDS (Urodynamic Study) is needed.

4. The method of claim 2, wherein the LUTS prediction model further comprises a first neural network for predicting a degree of BOO and a second neural network for predicting the degree of DUA.

5. The method of claim 4, wherein the LUTS prediction model is formed so that the second neural network has an output of the first neural network as an input value thereof, or the first neural network has an output of the second neural network as an input value thereof.

6. The method of claim 2, wherein the machine learning algorithm comprises any one of ANN (Artificial Neural Network), stacked auto-encoder, DNN (Deep Neural Network) and LSTM (Long Short Term Memory).

7. An LUTS diagnosis supporting system comprising:
a data input unit configured to receive urinary system data of an examinee;
a diagnosis prediction unit configured to derive prediction result data for the examinee by using an LUTS prediction model for the received urinary system data; and
a result providing unit configured to provide the derived prediction result data to a user terminal.

8. The LUTS diagnosis supporting system of claim 7, further comprising a prediction model generation unit configured to learn a correlation between diagnosis result data and urinary system data acquired in advance and stored in a database, through a machine learning algorithm, and generate an LUTS prediction model based on a result of the learning.

9. The LUTS diagnosis supporting system of claim 7, wherein the urinary system data comprises one or more of an age number of urinations, residual urine volume, uroflowmetry index, prostate symptom score, past medical history and voiding efficacy of the examinee,
wherein the prediction result data comprises one or more of predicted diagnosis, BOO (Bladder Outlet Obstruction) probability, DUA (Detrusor Under-Activity) probability, and information on whether UDS (Urodynamic Study) is needed.

10. The LUTS diagnosis supporting system of claim 8, wherein the LUTS prediction model further comprises a first neural network for predicting degree of BOO and a second neural network for predicting the degree of DUA.

11. The LUTS diagnosis supporting system of claim 10, wherein the LUTS prediction model is formed so that the second neural network has an output of the first neural network as an input value thereof, or the first neural network has an output of the second neural network as an input value thereof.

12. The LUTS diagnosis supporting system of claim 8, wherein the machine learning algorithm comprises any one of ANN (Artificial Neural Network), stacked auto-encoder, DNN (Deep Neural Network) and LSTM (Long Short Term Memory).

13. A computer readable recording medium in which a program for implementing the method of claim 1 is recorded.
